(19) United States
(12) Patent Application Publication
(10) Pub. No.: US 2024/0252517 A1
(43) Pub. Date: Aug. 1, 2024

(54) MINERALOCORTICOID RECEPTOR ANTAGONISTS FOR THE PREVENTION AND/OR TREATMENT OF A CORONAVIRUS INFECTION

(71) Applicant: Imaginatives Group Limited, London (GB)

(72) Inventor: Christopher Edwards, Kent (GB)

(21) Appl. No.: 18/565,721

(22) PCT Filed: Jun. 1, 2022
(86) PCT No.: PCT/GB22/51401

§ 371 Date: Nov. 30, 2023

(30) Foreign Application Priority Data
Jun. 1, 2021 (GB) 2107825.8

(51) Int. Cl. A61K 31/585; A61P 11/00; A61P 31/14

(52) U.S. Cl. CPC A61K 31/585; A61P 11/00; A61P 31/14

(57) ABSTRACT

The present invention relates to a novel therapy for preventing viral spread and/or reducing viral load in a patient and, in particular, a novel therapy for preventing viral spread and/or reducing viral load in a patient suffering with COVID-19. The therapy involves the administration of a mineralocorticoid receptor antagonist such as, for example, spironolactone.

### FIELD OF THE INVENTION

The present invention relates to a novel therapy for preventing viral spread and/or reducing viral load in a patient and, in particular, a novel therapy for preventing viral spread and/or reducing viral load in patients suffering with COVID-19.

### BACKGROUND OF THE INVENTION

It is well documented that the COVID-19 (SARS-CoV-2) pandemic is causing significant morbidity and mortality across the world, putting healthcare systems, and intensive care units in particular, under considerable strain. Vaccines provide promise for an end to the pandemic. However, it is becoming increasingly clear that a combination of new virus variants, rare but concerning vaccine complications, vaccine phobia and global production and logistical problems mean that complementary approaches to treating patients with the virus and for preventing the spread of the virus are going to be needed.

There is therefore a need for novel approaches for the treatment of coronavirus infections, such as COVID-19. In particular, there is a need for therapeutic approaches that can reduce the spread of the virus, by preventing the spread of the virus within an infected patient, thereby minimising their viral load, as well as diminishing the chance of virus being transmitted to other uninfected healthy individuals.

The present invention was devised with the foregoing in mind.

### SUMMARY OF THE INVENTION

The present invention resides in the hypothesis that both the COVID-19 (SARS-CoV-2) virus and ATP leave infected cells by means of lysosomal exocytosis, which is consequent on activation of the Mineralocorticoid Receptor (MR). The virus then is able to spread and infect other cells within the infected individual and transmit to other (uninfected) people. The release of ATP is implicated as a causative factor in the characteristic symptoms of a persistent cough and/or anosmia/ageusia experienced by many people suffering with COVID-19. The infection of endothelial cells expressing ACE2 results in the same process with exocytosis of Weibel-Palade bodies (WPB) containing the Von Willebrand Factor (responsible for micro-clotting) and angiopoietin 2 which increases vascular permeability and plays a major role in the Acute Respiratory Distress Syndrome (ARDS).

The present invention further resides in the recognition that the use of agents that inhibit lysosomal exocytosis could prevent the release of the virus and ATP from infected cells, thereby reducing the transmission of the virus between cells of the infected individual, as well as reducing transmission to other people. This will also serve to reduce the viral load within an infected individual. Blocking the release of ATP also assist in alleviating common symptoms of COVID-19 infection, including anosmia, ageusia and persistent cough experienced by many patients.

The present invention further resides in the recognition that this could be achieved by administering MR antagonists. The therapy of the present invention is anticipated to be especially useful in patients in the early stages of a COVID-19 infection, or a patient experiencing mild symptoms or asymptomatic disease. The MR antagonist may be given alone as the sole treatment, especially in the early stages on an infection. The MR antagonist may optionally be administered either orally and/or intranasally. In patients presenting with symptoms of a COVID-19 infection, and depending on the severity of the symptoms, the MR antagonist therapy may need to be combined with other therapeutic agents, e.g. a steroid such as dexamethasone given either orally or intranasally.

In one aspect, the present invention relates to a mineralocorticoid receptor antagonist, or a pharmaceutically acceptable salt thereof, for use in the prevention and/or treatment of a coronavirus infection (e.g. COVID-19). In an embodiment, the mineralocorticoid receptor antagonist, or a pharmaceutically acceptable salt thereof, is for use in:
(i) the prevention of a coronavirus infection (e.g. COVID-19) taking hold in an uninfected individual (i.e. a prophylactic use);
(ii) the treatment of a patient in the early stages of a COVID-19 infection, or a patient experiencing mild symptoms or a patient that is asymptomatic.
   Optionally, the mineralocorticoid receptor antagonist is used as the sole therapy or to prevent a healthy individual from developing an infection.

In another aspect, the present invention relates to a mineralocorticoid receptor antagonist, or a pharmaceutically acceptable salt thereof, for use in the prevention and/or treatment of a coronavirus infection (e.g. COVID-19), wherein the mineralocorticoid receptor antagonist, or a pharmaceutically acceptable salt thereof, reduces the viral load in the patient.

In another aspect, the present invention relates to a mineralocorticoid receptor antagonist, or a pharmaceutically acceptable salt thereof, for use in the prevention and/or treatment of a coronavirus infection (e.g. COVID-19), wherein the mineralocorticoid receptor antagonist, or a pharmaceutically acceptable salt thereof, reduces the transmission of virus.

In another aspect, the present invention relates to a mineralocorticoid receptor antagonist, or a pharmaceutically acceptable salt thereof, for use in the prevention of viral transmission in an individual infected with a virus (such as a coronavirus infection (e.g. COVID-19).

In another aspect, the present invention relates to a mineralocorticoid receptor antagonist, or a pharmaceutically acceptable salt thereof, for use in the reduction of the viral load in an individual infected with a virus (such as a coronavirus infection (e.g. COVID-19).

In another aspect, the present invention provides a mineralocorticoid receptor antagonist, or a pharmaceutically acceptable salt thereof, for use in the prevention and/or treatment of pneumonia in a patient suffering with a coronavirus (e.g. COVID-19) infection.

In another aspect, the present invention also relates to a mineralocorticoid receptor antagonist, or a pharmaceutically acceptable salt thereof, for use in the prevention and/or treatment of anosmia, ageusia (loss of taste), and/or persistent cough, in a patient suffering with anosmia, ageusia (loss of taste), and/or persistent cough (e.g. as a consequence of a coronavirus/COVID-19 infection).

As indicated above, and without wishing to be bound by any particular theory, the blocking the mineralocorocoid receptor with a mineralocorticoid receptor antagonist reduces lysosmal exocytosis of virus from an infected cell, thereby reducing the viral load and inhibiting the transmission of the virus to other cells in the infected individual and to other people.

In another aspect, the present invention relates to the use of a mineralocorticoid receptor antagonist, or a pharmaceutically acceptable salt thereof, in the manufacture of a medicament for use in the prevention and/or treatment of a coronavirus infection (e.g. COVID-19). In an embodiment, the mineralocorticoid receptor antagonist, or a pharmaceutically acceptable salt thereof, is for use in:
(i) the prevention of a coronavirus infection (e.g. COVID-19) taking hold in an uninfected individual (i.e. a prophylactic use);
(ii) the treatment of a patient in the early stages of a COVID-19 infection, or a patient experiencing mild symptoms or a patient that is asymptomatic.
   Optionally, the mineralocorticoid receptor antagonist is used as the sole therapy or to prevent a healthy individual from developing an infection.

In another aspect, the present invention relates to the use of a mineralocorticoid receptor antagonist, or a pharmaceutically acceptable salt thereof, in the manufacture of a medicament for use in the prevention and/or treatment of a coronavirus infection (e.g. COVID-19), wherein the mineralocorticoid receptor antagonist, or a pharmaceutically acceptable salt thereof, reduces the viral load in the patient.

In another aspect, the present invention relates to the use of a mineralocorticoid receptor antagonist, or a pharmaceutically acceptable salt thereof, in the manufacture of a medicament for use in the prevention and/or treatment of a coronavirus infection (e.g. COVID-19), wherein the mineralocorticoid receptor antagonist, or a pharmaceutically acceptable salt thereof, reduces the transmission of virus.

In another aspect, the present invention relates to the use of a mineralocorticoid receptor antagonist, or a pharmaceutically acceptable salt thereof, in the manufacture of a medicament for use in the prevention of viral transmission in an individual infected with a virus (such as a coronavirus infection (e.g. COVID-19).

In another aspect, the present invention relates to the use of a mineralocorticoid receptor antagonist, or a pharmaceutically acceptable salt thereof, in the manufacture of a medicament for use in the reduction of the viral load in an individual infected with a virus (such as a coronavirus infection (e.g. COVID-19).

In another aspect, the present invention provides the use of a mineralocorticoid receptor antagonist, or a pharmaceutically acceptable salt thereof, in the manufacture of a medicament for use in the prevention and/or treatment of pneumonia in a patient suffering with a coronavirus (e.g. COVID-19) infection.

In another aspect, the present invention also relates to the use of a mineralocorticoid receptor antagonist, or a pharmaceutically acceptable salt thereof, in the manufacture of a medicament for use in the prevention and/or treatment of anosmia, ageusia (loss of taste), and/or persistent cough, in a patient suffering with anosmia, loss of taste, and/or persistent cough (e.g. as a consequence of a coronavirus/COVID-19 infection).

In another aspect, the present invention relates to a method of preventing and/or treating a coronavirus infection (e.g. COVID-19), the method comprising administering a therapeutically effective amount of a mineralocorticoid receptor antagonist, or a pharmaceutically acceptable salt thereof, to a patient in need of such treatment. In an embodiment, the method is:
(i) a method of preventing a coronavirus infection (e.g. COVID-19) taking hold in an uninfected individual (i.e. a prophylactic use);
(ii) a method of treating a patient in the early stages of a COVID-19 infection, or a patient experiencing mild symptoms or a patient that is asymptomatic.
   Optionally, the mineralocorticoid receptor antagonist is used as the sole therapy or to prevent a healthy individual from developing an infection.

In another aspect, the present invention relates to a method of preventing and/or treating a coronavirus infection (e.g. COVID-19), the method comprising administering a therapeutically effective amount of a mineralocorticoid receptor antagonist, or a pharmaceutically acceptable salt thereof, to a patient in need of such treatment and wherein the mineralocorticoid receptor antagonist, or a pharmaceutically acceptable salt thereof, reduces the viral load in the patient.

In another aspect, the present invention relates to a method of preventing and/or treating a coronavirus infection (e.g. COVID-19), the method comprising administering a therapeutically effective amount of a mineralocorticoid receptor antagonist, or a pharmaceutically acceptable salt thereof, to a patient in need of such treatment and wherein the mineralocorticoid receptor antagonist, or a pharmaceutically acceptable salt thereof, reduces the transmission of virus.

In another aspect, the present invention relates to a method of preventing viral transmission in an individual infected with a virus (such as a coronavirus infection (e.g. COVID-19), the method comprising administering a therapeutically effective amount of a mineralocorticoid receptor antagonist, or a pharmaceutically acceptable salt thereof, to a patient in need of such treatment.

In another aspect, the present invention relates to a method of reducing viral load in an individual infected with a virus (such as a coronavirus infection (e.g. COVID-19), the method comprising administering a therapeutically effective amount of a mineralocorticoid receptor antagonist, or a pharmaceutically acceptable salt thereof, to a patient in need of such treatment.

In another aspect, the present invention relates to a method of treating or preventing pneumonia in an individual infected with a virus (such as a coronavirus infection (e.g. COVID-19), the method comprising administering a therapeutically effective amount of a mineralocorticoid receptor antagonist, or a pharmaceutically acceptable salt thereof, to a patient in need of such treatment.

In another aspect, the present invention also relates to method of preventing and/or treating anosmia, ageusia (loss of taste), and/or persistent cough, in a patient suffering with anosmia, ageusia (loss of taste), and/or persistent cough (e.g. as a consequence of a coronavirus/COVID-19 infection), the method comprising administering a therapeutically effective amount of a mineralocorticoid receptor antagonist, or a pharmaceutically acceptable salt thereof, to a patient in need of such treatment.

In another aspect, the present invention relates to a pharmaceutical composition comprising a mineralocorticoid receptor antagonist, or a pharmaceutically acceptable salt thereof, and one or more pharmaceutically acceptable excipients, and wherein the pharmaceutical composition is adapted for nasal and/or buccal delivery of the mineralocorticoid receptor antagonist. Optionally, the pharmaceutical compositions is in the form of a nasal spray, nasal drops, an oral/mouth spray, buccal tablet or mouthwash.

Preferred, suitable, and optional features of any one particular aspect of the present invention described herein are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

References to “a pharmaceutically acceptable salt” of an inhibitor defined herein is refers to any salt form suitable for pharmaceutical use. Examples of pharmaceutically acceptable salts include an acid-addition salt of an inhibitor of the invention which is sufficiently basic, for example, an acid-addition salt with, for example, an inorganic or organic acid, for example hydrochloric, hydrobromic, sulfuric, phosphoric, trifluoracetic, formic, citric methane sulfonate or maleic acid. In addition, a suitable pharmaceutically acceptable salt of an inhibitor of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

The mineralocorticoid receptor antagonist may also be administered in the form of a prodrug which is broken down in the human or animal body to release the active inhibitor. Examples of pro-drugs include in vivo cleavable ester derivatives of the inhibitors that may be formed at a carboxy group or a hydroxy group in an inhibitor compound and in-vivo cleavable amide derivatives that may be formed at a carboxy group or an amino group in an inhibitor compound. Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

As used herein the, “subject(s)” and/or “patient(s)”, suitably refer to human(s).

### Mineralocorticoid Receptor Antagonists

Mineralocorticoid receptor antagonists (also known as aldosterone antagonists) of the present invention are well known in the art.

Suitably, the mineralocorticoid receptor antagonist is selected from spironolactone, eplerenone or canrenone, or pharmaceutically acceptable salts of these agents. Most suitably, the mineralocorticoid receptor antagonist is spironolactone.

In a particular embodiment of the present invention, the mineralocorticoid receptor antagonist is spironolactone, or a pharmaceutically acceptable salt thereof.

In another particular embodiment of the present invention, the mineralocorticoid receptor antagonist is eplerenone, or a pharmaceutically acceptable salt thereof.

In another particular embodiment of the present invention, the mineralocorticoid receptor antagonist is canrenone, or a pharmaceutically acceptable salt thereof.

### Spironolactone

Spironolactone is a well known non-selective mineralocorticoid receptor antagonist, which has been used for many years for the treatment of conditions such as oedema, ascites, nephoritic syndrome, heart failure and resistant hypertension.

Suitable dosage ranges and schedules for the administration of spironolactone are known in the art.

For example, spironolactone may be administered at a dosage within the range of 25 mg-500 mg per day.

In the combination therapy of the present invention, the dose of spironolactone is suitably within the range of 25 mg to 400 mg per day. More suitably, the dosage of spironolactone is within the range of 25 mg to 200 mg per day.

In an embodiment of the invention, the dosage of spironolactone is within the range of 25 mg to 400 mg per day.

In an embodiment of the invention, the dosage of spironolactone is within the range of 50 mg to 400 mg per day.

In an embodiment of the invention, the dosage of spironolactone is within the range of 25 mg to 200 mg per day.

In an embodiment of the invention, the dosage of spironolactone is within the range of 50 mg to 200 mg per day.

In an embodiment of the invention, the dosage of spironolactone is 50 to 150 mg per day.

In an embodiment of the invention, the dosage of spironolactone is 100 to 150 mg per day.

In an embodiment of the invention, the dosage of spironolactone is 100 mg per day.

The daily dose of spironolactone may be administered as a single dose or as divided doses.

In an embodiment of the invention, the spironolactone is administered as a single daily dose.

Alternatively, spironolactone is administered in divided dosages, e.g.:
25 mg to 400 mg given twice daily;
50 mg to 400 mg given twice daily;
25 mg to 200 mg given twice daily;
50 mg to 200 mg given twice daily;
50 mg to 150 mg given twice daily;
100 mg to 200 mg given twice daily; or
100 mg to 150 mg given twice daily.

In an embodiment of the invention, spironolactone is administered at an initial loading dosage of 50 mg to 400 mg followed by a maintenance dosage of 25 mg to 200 mg twice daily.

In another embodiment of the invention, spironolactone is administered at an initial loading dosage of 50 mg to 200 mg followed by a maintenance dosage of 25 mg to 100 mg twice daily.

In another embodiment of the invention, spironolactone is administered at an initial loading dosage of 50 mg to 200 mg followed by a maintenance dosage of 25 mg to 100 mg twice daily.

In another embodiment of the invention, spironolactone is administered at an initial loading dosage of 50 mg to 150 mg followed by a maintenance dosage of 25 mg to 75 mg twice daily.

In another embodiment of the invention, spironolactone is administered at an initial loading dosage of 100 mg followed by a maintenance dosage of 50 mg twice daily.

Spironolactone is given orally, intra-nasally or by buccal delivery.

Treatment will be typically continued for between 1 and 6 weeks depending on the patient's symptoms. More typically, treatment will be continued for 1 to 3 weeks or 1 to 2 weeks.

### Eplerenone

Eplerenone is a selective mineralocorticoid receptor antagonist, which is indicated for use in the treatment of hypertension and heart failure.

Suitable dosage ranges and schedules for the administration of eplerenone are known in the art.

For example, eplerenone may be administered at a dosage within the range of 10 mg-100 mg per day.

In the combination therapy of the present invention, the dose of eplerenone is suitably within the range of 25 mg to 100 mg per day. More suitably, the dosage of eplerenone will be within the range of 25 mg to 50 mg per day.

In an embodiment of the invention, the dosage of eplerenone will be within the range of 25 mg to 100 mg per day.

In an embodiment of the invention, the dosage of eplerenone will be within the range of 25 mg to 50 mg per day.

In an embodiment of the invention, the dosage of eplerenone will be 25 mg per day.

In an embodiment of the invention, the dosage of eplerenone will be 50 mg per day.

In an embodiment of the invention, the dosage of eplerenone will be 100 mg per day.

The daily dose of eplerenone may be administered as a single dose or as divided doses.

In an embodiment of the invention, the eplerenone is administered as a single daily dose, e.g. a single daily dose of 25 mg or 50 mg per day.

In another embodiment of the invention, the eplerenone is administered in divided doses, e.g. a dose of 25 mg or 50 mg given twice daily.

In an embodiment of the invention, eplerenone is administered at an initial dosage of 25 mg or 50 mg once a day and this titrated to a maintenance dosage of 25 mg once or twice daily.

In another embodiment of the invention, eplerenone is administered at an initial dosage of 50 mg once a day and this titrated to a maintenance dosage of 25 mg once daily.

In another embodiment of the invention, eplerenone is administered at an initial dosage of 25 mg once a day and this titrated to a maintenance dosage of 50 mg once daily.

Suitably, the initial dosage is administered for 3 to 10 days, most suitably for a week.

Eplerenone is given orally, intra-nasally or by buccal delivery.

Treatment will be typically continued for between 1 and 6 weeks depending on the patient's symptoms. More typically, treatment will be continued for 1 to 3 weeks or 1 to 2 weeks.

### Canrenone

Canrenone is a metabolite of spironolactone which is used as a diuretic in a limited number of countries.

Suitable dosage ranges and schedules for the administration of canrenone are known in the art.

Canrenone can be given parenterally (e.g. intravenously), orally, intra-nasally or by buccal delivery.

Treatment will be typically continued for between 1 and 6 weeks depending on the patient's symptoms. More typically, treatment will be continued for 1 to 3 weeks or 1 to 2 weeks.

### Rationale for the Therapy of the Present Invention

Without wishing to be bound by any particular theory, the rationale behind the combination therapy of the present invention and the basis for the original hypothesis behind this therapy is set out below:

### DESCRIPTION OF FIGURES

FIG. 1—SARS-CoV-2 virus binds to ACE2 receptor and uses this as a way of getting into the cell.

FIG. 2—Release of ATP from basolateral aspect of renal epithelial cells in response to activation of Mineralocorticoid Receptor. ATP than acts on local purinergic receptors to increase calcium entry. This then results in cell contraction with opening of the apical sodium channel.

FIG. 3—Inhibition of exocytosis of Von Willebrand Factor from Weibel-Palade bodies in endothelial cells and its inhibition by spironolactone (Jeong et al. 2009 PNAS)⁴.

FIG. 4—Entry and exit of SARS-CoV-2 from the cell. The virus hijacks lysosomes to get out of the cell.

FIG. 5—pH dependence of lysosomal exocytosis (Miao et al. 2015). Deacidification of the lysosome results in exocytosis of Uro-Pathogenic E. coli (UPEC).

FIG. 6—Key cells in nasal epithelium.

FIG. 7—shows the results of Ukraine study 1 described in example 1. Clinical findings (%) in Group I given high dose dexamethasone (HIDEX) compared to low dose dexamethasone and spironolactone (SPIDEX) at baseline (lower bars) and after 5 days (upper bars).

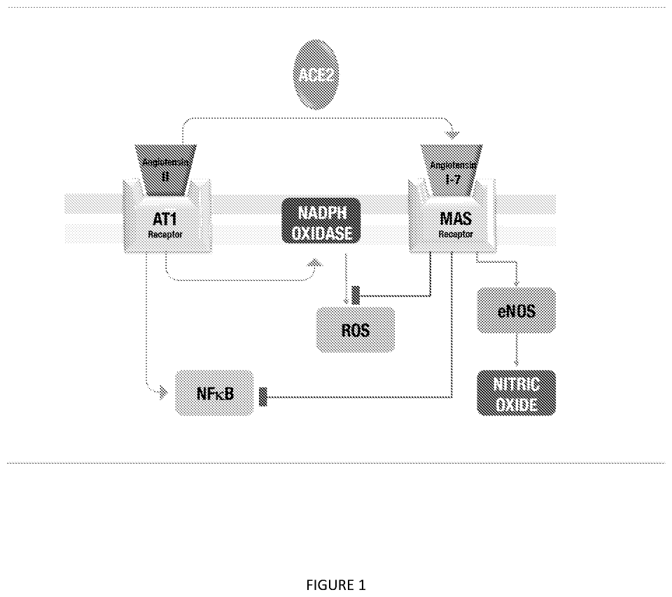

COVID-19 (SARS-CoV-2) virus binds to ACE2 receptor on specific cells types (e.g. nasal sustentacular cells)¹ [see FIG. 1 of the accompanying drawings]. The virus destroys ACE2 and this results in the loss of local conversion of angiotensin II to angiotensin¹⁻⁷. This leads to the consequent stimulation of NADPH oxidase by angiotensin II and an increase Reactive Oxygen Species (ROS). Consequent DNA damage and activation of DNA repair enzyme Poly ADP Ribose Polymerase (PARP) then occurs. PARP uses NAD as a co-factor and excessive PARP produces NAD deficiency (e.g. the elderly have increasing PARP activity and progressive decrease in NAD). NAD is the co-factor used by enzyme 11β-HSD2 that inactivates cortisol by converting it to cortisone. This is the mechanism that protects the Mineralocorticoid Receptor from being activated by cortisol and makes it aldosterone selective². Loss of this protection in cells infected by the virus allows local cortisol to control the MR activation.

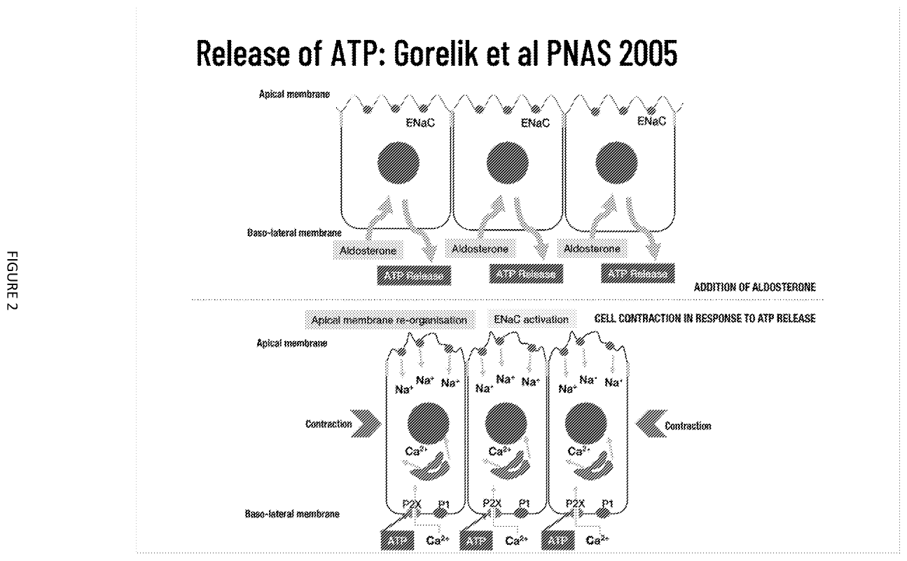

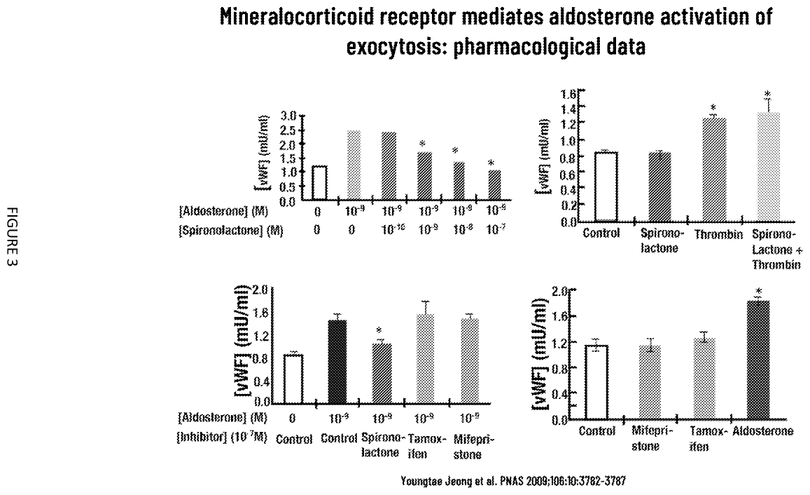

MR activation produces release of ATP from the basolateral part of the cell. This then acts on purinergic receptors that are located there and results in an increase in intracellular calcium³. The rise in calcium leads to apical cell contraction with associated opening of the sodium channel and exocytosis of lysosomes from the cell [see FIG. 2]. In endothelial cells these are Weibel-Palade bodies, the lysosome-related secretory organelles of these cells. Jeong et al. first demonstrated this and showed that exocytosis could be blocked by spironolactone⁴.

Oxidative stress has been shown to increase the exocytosis of lysosomes containing ATP⁵. This mechanism requires an increase in intracellular calcium which could be due to the MR mechanism described above.

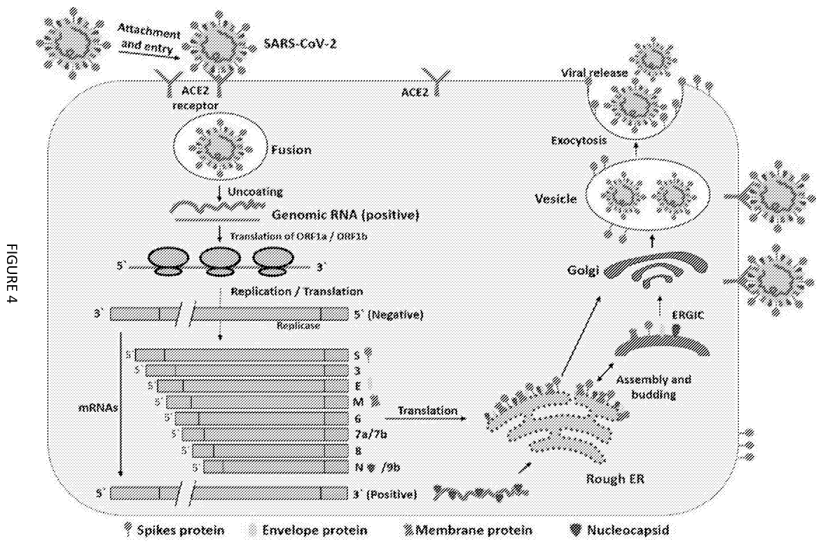

It has been demonstrated that the coronavirus exits cells by hijacking the lysosomal pathway⁶ [see FIG. 4]. In this process the normally acidic lysosomes are de-acidified. This change in pH markedly stimulates this exocytic pathway. The reason for the change in pH is not clear but studies have shown that angiotensin II regulates the Sodium/Hydrogen Exchanger⁷. The loss of protons from the cell in exchange for sodium raises intracellular pH. The NHE proteins 1-5 are on the cell surface. Intracellularly there is a subfamily of NHE 6-9 that are localised to the membranes of organelles including late endosomes which fuse with lysosomes. Studies have shown that lysosomal deacidification is key for the opening of the pH dependent calcium channel in the wall of the lysosomes⁸. This channel, TRPML3, is only active at neutral pH. Entry of calcium into the lysosome stimulates exocytosis.

Lysosomal exocytosis produced by this mechanism then results in:
Release of the virus which can then infect other ACE2 containing cells.
Release of ATP which then produces the characteristic features of COVID-19, loss of taste/small due to the purinergic nociceptive response, the characteristic non-productive cough and release of the inflammasome with consequent inflammatory response.
In endothelial cells the release of the Weibel-Palade bodies results in high circulating levels of Von Willebrand Factor with consequent microthrombi, and angiopoietin 2 which increases vascular permeability and leads to the Acute Respiratory Distress Syndrome (ARDS) responsible for most of the deaths from SARS-CoV-2 infection.
The long-term loss of ACE2 (possibly in some patients by producing autoantibodies to ACE2) results in many of the key features of Long COVID such as chronic fatigue (possibly due to chronic ATP depletion).

Early results of studies using a combination of spironolactone to block the Mineralocorticoid Receptor and dexamethasone to suppress cortisol and thus prevent it acting as an MR agonist have been very encouraging (see Example 1). In 40 patients from the Ukraine with pneumonia given low dose dexamethasone and spironolactone all clinical features, biochemistry (CRP and D-Dimer) and serial chest X-rays were superior to those found in 40 matched patients with pneumonia given conventional therapy with high-dose dexamethasone. There was only 1 death and that was in the high-dose dexamethasone group.

In addition, a study published from Milan using the spironolactone metabolite canrenone which can be given intravenously was even more encouraging⁹. They looked at patients with COVID-19 admitted to hospital with moderate to severe respiratory failure. In group A given conventional treatment (which included methylprednisolone (i.e. a steroid)) they had a 36% mortality. In group B given canrenone the mortality was 13%. In group A 49% had to go onto a ventilator. In group B only 20% required ventilation. The D-Dimer levels rose significantly in those in the control group and fell in the canrenone group.

The numbers in this study were small and it was not randomised. It is therefore difficult to compare with the results of the RECOVERY trial of dexamethasone¹⁰. In the RECOVERY trial control group they had a 24.6% mortality as compared to 21.6% in those given dexamethasone.

### The Role of the Nasal Epithelium

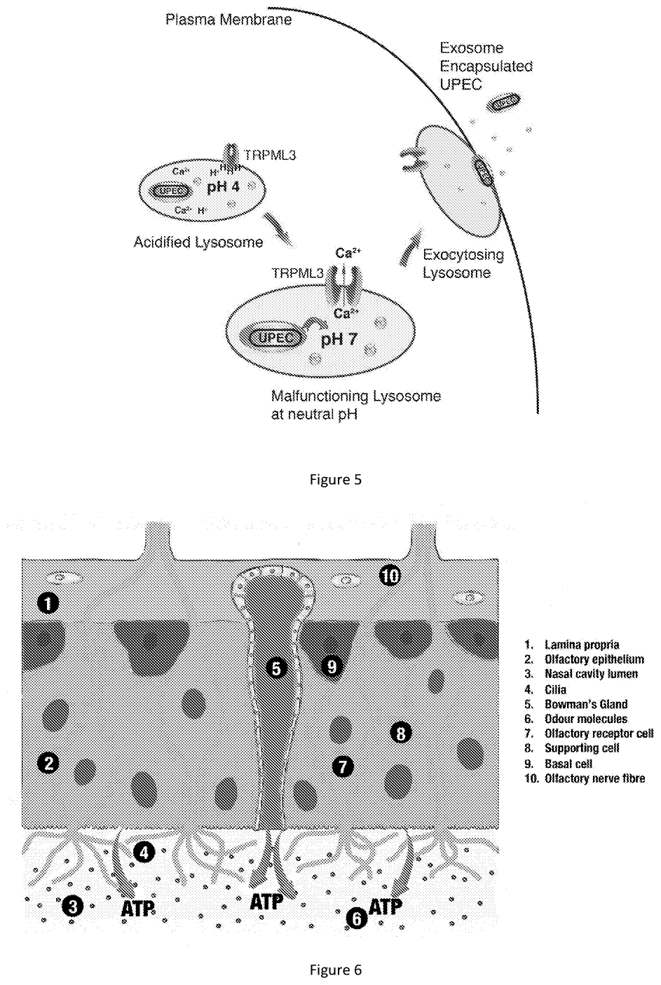

In reference to FIG. 6, when the virus enters the nose it binds to ACE2 receptors which are on the supporting cells (8) and the cells lining Bowman's gland (5). If the hypothesis is correct then the Mineralocorticoid Receptors in these cells are activated by cortisol. This then leads to the basolateral release of ATP. This activates local purinergic receptors with consequent entry of calcium into the cells. Calcium-induced calcium release then stimulates exocytosis of ATP and the virus from the apical membranes of infected cells.

ATP then acts on the purinergic receptors of the olfactory receptor cells (7). The high concentration of ATP then produces a nociceptive response with consequent anosmia. Inhibition of exocytosis by MR blockade should lower nasal ATP levels with recovery of the sense of smell.

In the Ukraine study described in the example section, 85% of patients in the spironolactone/low dose dexamethasone group had loss of smell/taste at baseline. After 5 days of treatment this had reduced to 47.5%. This compared to high dose dexamethasone which had loss of taste/small in 82.5% at baseline and 75% after 5 days treatment. This implies the spironolactone played a role in inhibiting lysosomal

In the same way inhibition of exocytosis should lead to a reduction of viral release into the nose.

### REFERENCES

1. Edwards, C. New Horizons: Does Mineralocorticoid Receptor Activation by Cortisol Cause ATP Release and COVID-19 Complications? J. Clin. Endocrinol. Metab. XX, 1-14 (2020).
2. Edwards, C. R. W. et al. Localisation of 11B-Hydroxysteroid Dehydrogenase-Tissue Specific Protector of the Mineralocorticoid Receptor. Lancet 332, 986-989 (1988).
3. Gorelik, J. et al. Aldosterone acts via an ATP autocrine/paracrine system: the Edelman ATP hypothesis revisited. Proc. Natl. Acad. Sci. U.S.A. 102, 15000-5 (2005).
4. Jeong, Y. et al. Aldosterone activates endothelial exocytosis. Proc. Natl. Acad. Sci. U.S.A. 106, 3782-3787 (2009).
5. Li, Z. et al. Lysosome exocytosis is involved in astrocyte ATP release after oxidative stress induced by H2O2. Neuroscience Letters 705, 251-258 (2019).
6. Ghosh, S. et al. β-Coronaviruses Use Lysosomes for Egress Instead of the Biosynthetic Secretory Pathway. Cell 183, 1520-1535.e14 (2020).
7. Peti-peterdi, J. & Bell, P. D. Regulation of Macula densa Na:H exchange by angiotensin II. Kidney International 54, 2021-2028 (1998).
8. Miao, Y., Li, G., Zhang, X., Xu, H. & Abraham, S. N. A TRP channel senses lysosome neutralization by pathogens to trigger their expulsion. Cell 161, 1306-1319 (2015).
9. Vicenzi, M. et al. The Efficacy of the Mineralcorticoid Receptor Antagonist Canrenone in COVID-19 Patients. J. Clin. Med. 9, 2943 (2020).
10. Dexamethasone in Hospitalized Patients with Covid-19—Preliminary Report. N. Engl. J. Med. 1-11 (2020). doi:10.1056/nejmoa2021436

### Therapeutic Uses

The present invention relates to the use of a mineralocorticoid receptor antagonist (e.g. spironoloctone) in the treatment of a coronavirus (e.g. COVID-19) infection. In particular, a mineralocorticoid receptor antagonist (e.g. spironoloctone) prevents the transmission of coronavirus by blocking the mineralocorticoid receptor and inhibiting mineralocorticoid receptor-induced lysosomal exocytosis, which is postulated to be a mechanism by which the virus transmits from an infected cell.

The inhibition of lysosomal exocytosis also reduces the release of ATP which is implicated in the common symptoms of anosmia, ageusia and persistent cough observed in COVID-19 patients.

In one aspect, the present invention relates to a mineralocorticoid receptor antagonist, or a pharmaceutically acceptable salt thereof, for use in the prevention and/or treatment of a coronavirus infection (e.g. COVID-19).

In another aspect, the present invention relates to a mineralocorticoid receptor antagonist, or a pharmaceutically acceptable salt thereof, for use in the prevention and/or treatment of a coronavirus infection (e.g. COVID-19), wherein the mineralocorticoid receptor antagonist, or a pharmaceutically acceptable salt thereof, reduces the viral load in the patient.

In another aspect, the present invention relates to a mineralocorticoid receptor antagonist, or a pharmaceutically acceptable salt thereof, for use in the prevention and/or treatment of a coronavirus infection (e.g. COVID-19), wherein the mineralocorticoid receptor antagonist, or a pharmaceutically acceptable salt thereof, reduces the transmission of virus.

In another aspect, the present invention relates to a mineralocorticoid receptor antagonist, or a pharmaceutically acceptable salt thereof, for use in the prevention of viral transmission in an individual infected with a virus (such as a coronavirus infection (e.g. COVID-19).

In another aspect, the present invention relates to a mineralocorticoid receptor antagonist, or a pharmaceutically acceptable salt thereof, for use in the reduction of the viral load in an individual infected with a virus (such as a coronavirus infection (e.g. COVID-19).

In another aspect, the present invention provides a mineralocorticoid receptor antagonist, or a pharmaceutically acceptable salt thereof, for use in the prevention and/or treatment of pneumonia in a patient suffering with a coronavirus (e.g. COVID-19) infection.

In another aspect, the present invention also relates to a mineralocorticoid receptor antagonist, or a pharmaceutically acceptable salt thereof, for use in the prevention and/or treatment of anosmia, ageusia (loss of taste), and/or persistent cough, in a patient suffering with anosmia, ageusia (loss of taste), and/or persistent cough (e.g. as a consequence of a coronavirus/COVID-19 infection).

In another aspect, the present invention relates to a mineralocorticoid receptor antagonist, or a pharmaceutically acceptable salt thereof, for use in the prevention and/or treatment of a coronavirus infection (e.g. COVID-19), wherein the mineralocorticoid receptor antagonist is administered either orally or locally to the nasal epithelium and/or the buccal cavity.

In another aspect, the present invention relates to a mineralocorticoid receptor antagonist, or a pharmaceutically acceptable salt thereof, for use in the prevention and/or treatment of a coronavirus infection (e.g. COVID-19), wherein the mineralocorticoid receptor antagonist, or a pharmaceutically acceptable salt thereof, reduces the viral load in the patient and wherein the mineralocorticoid receptor antagonist is administered either orally or locally to the nasal epithelium and/or the buccal cavity.

In another aspect, the present invention relates to a mineralocorticoid receptor antagonist, or a pharmaceutically acceptable salt thereof, for use in the prevention and/or treatment of a coronavirus infection (e.g. COVID-19), wherein the mineralocorticoid receptor antagonist, or a pharmaceutically acceptable salt thereof, reduces the transmission of virus and wherein the mineralocorticoid receptor antagonist is administered either orally or locally to the nasal epithelium and/or the buccal cavity.

In another aspect, the present invention relates to a mineralocorticoid receptor antagonist, or a pharmaceutically acceptable salt thereof, for use in the prevention of viral transmission in an individual infected with a virus (such as a coronavirus infection (e.g. COVID-19) and wherein the mineralocorticoid receptor antagonist is administered either orally or locally to the nasal epithelium and/or the buccal cavity.

In another aspect, the present invention relates to a mineralocorticoid receptor antagonist, or a pharmaceutically acceptable salt thereof, for use in the reduction of the viral load in an individual infected with a virus (such as a coronavirus infection (e.g. COVID-19), wherein the mineralocorticoid receptor antagonist is administered either orally or locally to the nasal epithelium and/or the buccal cavity.

In another aspect, the present invention also relates to a mineralocorticoid receptor antagonist, or a pharmaceutically acceptable salt thereof, for use in the prevention and/or treatment of:
(i) anosmia, wherein the mineralocorticoid receptor antagonist is administered either orally or locally to the nasal epithelium;
(ii) ageusia (loss of taste) wherein the mineralocorticoid receptor antagonist is administered either orally or locally to the buccal cavity; and/or
(iii) persistent cough, wherein the mineralocorticoid receptor antagonist is administered either orally or locally to the nasal epithelium and/or the buccal cavity; in a patient suffering with anosmia, ageusia (loss of taste), and/or persistent cough (e.g. as a consequence of a coronavirus/COVID-19 infection).

In another aspect, the present invention relates to the use of a mineralocorticoid receptor antagonist, or a pharmaceutically acceptable salt thereof, in the manufacture of a medicament for use in the prevention and/or treatment of a coronavirus infection (e.g. COVID-19).

In another aspect, the present invention relates to the use of a mineralocorticoid receptor antagonist, or a pharmaceutically acceptable salt thereof, in the manufacture of a medicament for use in the prevention and/or treatment of a coronavirus infection (e.g. COVID-19), wherein the mineralocorticoid receptor antagonist, or a pharmaceutically acceptable salt thereof, reduces the viral load in the patient.

In another aspect, the present invention relates to the use of a mineralocorticoid receptor antagonist, or a pharmaceutically acceptable salt thereof, in the manufacture of a medicament for use in the prevention and/or treatment of a coronavirus infection (e.g. COVID-19), wherein the mineralocorticoid receptor antagonist, or a pharmaceutically acceptable salt thereof, reduces the transmission of virus.

In another aspect, the present invention relates to the use of a mineralocorticoid receptor antagonist, or a pharmaceutically acceptable salt thereof, in the manufacture of a medicament for use in the prevention of viral transmission in an individual infected with a virus (such as a coronavirus infection (e.g. COVID-19).

In another aspect, the present invention relates to the use of a mineralocorticoid receptor antagonist, or a pharmaceutically acceptable salt thereof, in the manufacture of a medicament for use in the reduction of the viral load in an individual infected with a virus (such as a coronavirus infection (e.g. COVID-19).

In another aspect, the present invention provides the use of a mineralocorticoid receptor antagonist, or a pharmaceutically acceptable salt thereof, in the manufacture of a medicament for use in the prevention and/or treatment of pneumonia in a patient suffering with a coronavirus (e.g. COVID-19) infection.

In another aspect, the present invention also relates to the use of a mineralocorticoid receptor antagonist, or a pharmaceutically acceptable salt thereof, in the manufacture of a medicament for use in the prevention and/or treatment of anosmia, ageusia (loss of taste), and/or persistent cough, in a patient suffering with anosmia, ageusia (loss of taste), and/or persistent cough (e.g. as a consequence of a coronavirus/COVID-19 infection).

In another aspect, the present invention relates to the use of a mineralocorticoid receptor antagonist, or a pharmaceutically acceptable salt thereof, in the manufacture of a medicament for use in the prevention and/or treatment of a coronavirus infection (e.g. COVID-19), wherein the mineralocorticoid receptor antagonist is administered either orally or locally to the nasal epithelium and/or the buccal cavity.

In another aspect, the present invention relates to the use of a mineralocorticoid receptor antagonist, or a pharmaceutically acceptable salt thereof, in the manufacture of a medicament for use in the prevention and/or treatment of a coronavirus infection (e.g. COVID-19), wherein the mineralocorticoid receptor antagonist, or a pharmaceutically acceptable salt thereof, reduces the viral load in the patient, and wherein the mineralocorticoid receptor antagonist is administered either orally or locally to the nasal epithelium and/or the buccal cavity.

In another aspect, the present invention relates to the use of a mineralocorticoid receptor antagonist, or a pharmaceutically acceptable salt thereof, in the manufacture of a medicament for use in the prevention and/or treatment of a coronavirus infection (e.g. COVID-19), wherein the mineralocorticoid receptor antagonist, or a pharmaceutically acceptable salt thereof, reduces the transmission of virus, and wherein the mineralocorticoid receptor antagonist is administered either orally or locally to the nasal epithelium and/or the buccal cavity.

In another aspect, the present invention relates to the use of a mineralocorticoid receptor antagonist, or a pharmaceutically acceptable salt thereof, in the manufacture of a medicament for use in the prevention of viral transmission in an individual infected with a virus (such as a coronavirus infection (e.g. COVID-19), and wherein the mineralocorticoid receptor antagonist is administered either orally or locally to the nasal epithelium and/or the buccal cavity.

In another aspect, the present invention relates to the use of a mineralocorticoid receptor antagonist, or a pharmaceutically acceptable salt thereof, in the manufacture of a medicament for use in the reduction of the viral load in an individual infected with a virus (such as a coronavirus infection (e.g. COVID-19), and wherein the mineralocorticoid receptor antagonist is administered either orally or locally to the nasal epithelium and/or the buccal cavity.

In another aspect, the present invention also relates to the use of a mineralocorticoid receptor antagonist, or a pharmaceutically acceptable salt thereof, in the manufacture of a medicament for use in the prevention and/or treatment of:
(i) anosmia, wherein the mineralocorticoid receptor antagonist is administered either orally or locally to the nasal epithelium;
(ii) ageusia (loss of taste) wherein the mineralocorticoid receptor antagonist is administered either orally or locally to the buccal cavity; and/or
(iii) persistent cough, wherein the mineralocorticoid receptor antagonist is administered either orally or locally to the nasal epithelium and/or the buccal cavity;
   in a patient suffering with anosmia, ageusia (loss of taste), and/or persistent cough (e.g. as a consequence of a coronavirus/COVID-19 infection).

In another aspect, the present invention relates to a method of preventing and/or treating a coronavirus infection (e.g. COVID-19), the method comprising administering a therapeutically effective amount of a mineralocorticoid receptor antagonist, or a pharmaceutically acceptable salt thereof, to a patient in need of such treatment.

In another aspect, the present invention relates to a method of preventing and/or treating a coronavirus infection (e.g. COVID-19), the method comprising administering a therapeutically effective amount of a mineralocorticoid receptor antagonist, or a pharmaceutically acceptable salt thereof, to a patient in need of such treatment and wherein the mineralocorticoid receptor antagonist, or a pharmaceutically acceptable salt thereof, reduces the viral load in the patient.

In another aspect, the present invention relates to a method of preventing and/or treating a coronavirus infection (e.g. COVID-19), the method comprising administering a therapeutically effective amount of a mineralocorticoid receptor antagonist, or a pharmaceutically acceptable salt thereof, to a patient in need of such treatment and wherein the mineralocorticoid receptor antagonist, or a pharmaceutically acceptable salt thereof, reduces the transmission of virus.

In another aspect, the present invention relates to a method of preventing viral transmission in an individual infected with a virus (such as a coronavirus infection (e.g. COVID-19), the method comprising administering a therapeutically effective amount of a mineralocorticoid receptor antagonist, or a pharmaceutically acceptable salt thereof, to a patient in need of such treatment.

In another aspect, the present invention relates to a method of reducing viral load in an individual infected with a virus (such as a coronavirus infection (e.g. COVID-19), the method comprising administering a therapeutically effective amount of a mineralocorticoid receptor antagonist, or a pharmaceutically acceptable salt thereof, to a patient in need of such treatment.

In another aspect, the present invention relates to a method of treating or preventing pneumonia in an individual infected with a virus (such as a coronavirus infection (e.g. COVID-19), the method comprising administering a therapeutically effective amount of a mineralocorticoid receptor antagonist, or a pharmaceutically acceptable salt thereof, to a patient in need of such treatment.

In another aspect, the present invention also relates to method of preventing and/or treating anosmia, ageusia (loss of taste), and/or persistent cough, in a patient suffering with anosmia, ageusia (loss of taste), and/or persistent cough (e.g. as a consequence of a coronavirus/COVID-19 infection), the method comprising administering a therapeutically effective amount of a mineralocorticoid receptor antagonist, or a pharmaceutically acceptable salt thereof, to a patient in need of such treatment.

Suitably, the mineralocorticoid receptor antagonist is administered either orally or locally to the nasal epithelium and/or the buccal cavity. Most suitably, the mineralocorticoid receptor antagonist is administered to the nasal epithelium and/or buccal cavity.

### Pharmaceutical Compositions

The mineralocorticoid receptor antagonist is administered in the form a pharmaceutical composition comprising the mineralocorticoid receptor antagonist, or a pharmaceutically acceptable salt thereof, and one more pharmaceutically acceptable excipients.

In a particular embodiment of the invention, the pharmaceutical composition is suitable for oral delivery of the mineralocorticoid receptor antagonist. For example, it is in the form of a tablet, lozenges, hard or soft capsules, aqueous or oily suspension, emulsion, dispersible powder or granule, syrup or elixir.

In another particular embodiment of the invention, the pharmaceutical composition is suitable for nasal delivery of the mineralocorticoid receptor antagonist. For example, it is in the form of a nasal spray or nose drops.

In another particular embodiment of the invention, the pharmaceutical composition is suitable for buccal delivery of the mineralocorticoid receptor antagonist. For example, it is in the form of a buccal spray, mouthwash or buccal tablet.

Thus, in a particular aspect, the present invention provides a pharmaceutical composition comprising a mineralocorticoid receptor antagonist, or a pharmaceutically acceptable salt thereof, and one or more pharmaceutically acceptable excipients, and wherein the pharmaceutical composition is adapted for nasal and/or buccal delivery of the mineralocorticoid receptor antagonist.

The pharmaceutical compositions may be obtained by conventional procedures using conventional pharmaceutical excipients well known in the art. Thus, compositions intended for oral use may contain, for example, one or more colouring, sweetening, flavouring and/or preservative agents.

An effective amount of a mineralocorticoid receptor antagonist, or a pharmaceutically acceptable salt thereof, for use in accordance with the present invention is an amount sufficient to prevent viral transmission and/or reduce the viral load in a patient, as well as alleviate the symptoms of anosmia (loss of smell), ageusia (loss of taste) and/or cough (including persistent cough).

The amount of active ingredient that is combined with one or more excipients to produce a single dosage form will necessarily vary depending upon the individual treated and the particular route of administration. For example, a formulation intended for oral administration to humans will generally contain, for example, from 0.25 mg to 0.25 g of active agent (more suitably from 0.5 to 150 mg, for example from 1 to 100 mg) compounded with an appropriate and convenient amount of excipients which may vary from about 5 to about 98 percent by weight of the total composition.

The size of the dose for therapeutic or prophylactic purposes of a combination of the invention will naturally vary according to the nature and severity of the conditions, the age and sex of the animal or patient and the route of administration, according to well-known principles of medicine.

Combinations with Additional Therapeutic Agents

The mineralocorticoid receptor antagonist treatment defined herein may be applied as a sole therapy or it may involve, in addition to the mineralocorticoid receptor antagonist therapy of the present invention, one or more additional therapies (including treatment with another therapeutic agent or other therapeutic interventions).

Typically, the other therapeutic agent used in combination with the mineralocorticoid receptor antagonist therapy of the present invention will be one or more therapeutic agents used as the standard of care for the treatment of the disease or condition concerned or various symptoms thereof. The other therapeutic agent may include, for example, another drug used for the treatment of the condition concerned, or an agent that modulates the biological response to the mineralocorticoid receptor antagonist therapy of the invention.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

### EXAMPLES

### Example 1—Clinical Studies into Spironolactone/Dexamethasone Treatment of COVID-19

### Methods:

This study report presents the results of a proof of concept study in which a combination of spironolactone and dexamethasone has been used to block the mineralocorticoid receptor which has been activated by the virus. The study is in two parts. The first relates to 40 patients with COVID-19 admitted to hospital with pneumonia treated with conventional high dose dexamethasone (HIDEX) and antibiotic and compares this with 40 similar patients treated with spironolactone and low dose dexamethasone (SPIDEX) and antibiotic. In a second study SPIDEX was given to 20 COVID-positive outpatients without pneumonia within 2-3 days of first symptoms of disease.

### Findings:

On all parameters in study 1 (clinical, biochemical and radiological) SPIDEX was superior to HIDEX. In study 2 90% patients had resolution of symptoms and signs within 10 days of starting treatment. Only 2 required short hospital admission. Hyperkalaemia which has been suggested as a possible problem with this approach was not found.

### Interpretation:

These results suggest that it may be possible to safely treat COVID-19 patients with two cheap, readily available drugs with potential major health and economic benefit. This was an open study. Randomised trials of this treatment are underway.

### Introduction to Clinical Study

This study describes an approach which is based on increased understanding of the metabolic consequences of the SARS-CoV-2 viral infection.

A large number of studies have shown that the virus enters cells using the ACE2 receptor which is present on key epithelial cells such the nose, tongue, salivary glands and lungs. After the primary infection, patients with specific co-morbidities such as hypertension, cardiovascular disease and diabetes, are at risk of developing an endothelitis which plays a vital role in the development of the major complications of Acute Respiratory Distress Syndrome (ARDS) and microthrombi. Young people and those with normal microcirculation have very low expression of ACE2 receptors on their endothelial cells. In contrast those with vascular disease have high levels of expression induced by changes in blood flow, shear stress and change in basement membrane.

The loss of the ACE2 receptor results in a failure of the conversion of angiotensin II to angiotensin (1-7) (FIG. 1).

### Methods

### Patient Group Description, Treatments and Biochemical Testing

### Study 1

This was a retrospective study of 80 patients with PCR-confirmed infection with SARS-CoV-2 admitted to Vinnytsia State Hospital, Vinnytsia, Ukraine with a radiologically confirmed diagnosis of bilateral pneumonia. 40 patients (Group 1—HIDEX) were then given standard treatment with high dose dexamethasone (16 mg/day), a mucolytic ambroxol 90 mg/day and antibiotic ceftriaxone 2 g/day. A further 40 patients (Group II—SPIDEX) were given low dose dexamethasone 4 mg/day, spironolactone 100 mg/day, ambroxol 90 mg/day, ceftriaxone 2 g/day. Both groups received treatment for 10 days. The treatment was not based on a randomised design and both groups were composed a posteriori.

Before treatment and during their stay in hospital all patients had routine clinical assessment: body temperature, blood pressure, heart rate, respiratory rate, blood oxygen saturation: routine biochemistry, C-reactive protein (CRP), D-dimer, fasting blood glucose:radiology, baseline and day 5 chest X-rays.

The age range of patients treated was from 28-84 years.

The patient characteristics, co-morbidities, and presenting symptoms are listed in Table 1.

TABLE 1
Study 1. Patients' general characteristics,
co-morbidities and clinical parameters at baseline

| | | |
|---|---|---|
| Features | Group I HIDEX | Group II SPIDEX |

| | | |
|---|---|---|
| Number | 40 | 40 |

| | | | | |
|---|---|---|---|---|
| Women | 27 | (67.5%) | 25 | (62.5%) |
| Men | 13 | (32.5%) | 15 | (37.5%) |

| | | |
|---|---|---|
| Average age, years | 61.1 ± 12.3 | 61.4 ± 11.8 |

| | | | | |
|---|---|---|---|---|
| Hypertension | 26 | (65%) | 27 | (67.5%) |
| Ischemic heart disease | 24 | (60%) | 31 | (77.5%) |
| Diabetes | 10 | (25%) | 10 | (25%) |
| Obesity | 16 | (40%) | 19 | (47.5%) |
| Chronic kidney disease | 2 | (5%) | 3 | (7.5%) |
| Oncopathology | 1 | (2.5%) | 2 | (5%) |
| Loss of smell / taste | 33 | (82.5%) | 34 | (85%) |
| Sore throat | 22 | (55%) | 26 | (65%) |
| Breathlessness | 30 | (75%) | 28 | (70%) |
| Dry cough | 36 | (90%) | 32 | (89%) |
| Fever | 38 | (95%) | 37 | (92.5) |
| Need for oxygen | 22 | (55%) | 18 | (45%) |

### Study 2.

This was a study of the effectiveness and safety of treating 20 outpatients with PCR-confirmed COVID-19 patients. Treatment was started within 2-3 days of first symptoms. No patient had pneumonia. Dexamethasone was given in a dose of 4 mg/day, spironolactone 100 mg/day: both drugs were given for 10 days.

The age range of patients treated was 21-58 years.

The patient characteristics, co-morbidities, and presenting symptoms are listed in Table 2.

TABLE 2
Study 2. Patients' general characteristics,
co-morbidities and clinical parameters at baseline

| Features | Number of patients | |
|---|---|---|
| Women | 8 | (40%) |
| Men | 12 | (60%) |
| Average age, years | 44.8 ± 13.9 | |
| Need for hospitalization within 10 d | 2 | (10%) |
| Number of deaths in 10 days | 0 | |
| Co-morbidity | 6 | (30%) |

| | Number of patients |
|---|---|
| Concomitant pathology | (n = 6) |
| Hypertension | 5 |
| Diabetes | 2 |
| Obesity | 4 |
| Loss of smell / taste | 18 (90%) |
| Sore throat | 11 (55%) |
| Breathlessness | 10 (50%) |
| Dry cough | 16 (80%) |
| Fever | 16 (80%) |
| Blood oxygen < 92% | 0 |
| Body temperature ° C. | 37.8 ± 0.7 |
| Oxygen saturation (%) | 96.4 ± 1.7 |

### Patient and Other Consents

The studies received approval from the Vinnytsia Hospital Ethics Committee and were carried out with informed consent from participants.

### Statistical Method

For normally distributed clinical characteristics and demographics data were expressed as the mean and standard deviation.

### Findings

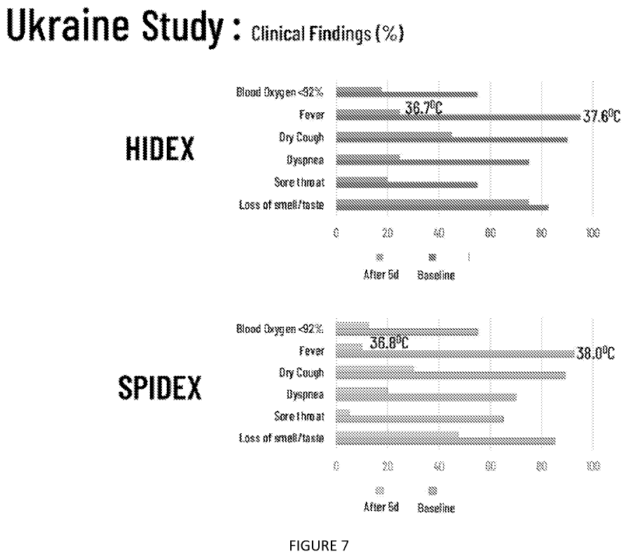

Study 1. The two groups did not significantly differ as judged by their age, sex, and clinical characteristics including their co-morbidities. Only 1 patient died: they were in group I given high dose dexamethasone (HIDEX). No patients required ventilation. Patients with oxygen saturation less than 92% were given oxygen by mask. The results of the clinical findings in the HIDEX and SPIDEX groups after 5 days of treatment are shown in FIG. 7.

Both treatment regimens produced clinical improvement but, as judged by every symptom and sign assessed at 5 days, this was greater on the SPIDEX than the HIDEX regimen. A strict comparison of the 2 groups at 10 days was not possible as more patients had been discharged before 10 days in the SPIDEX group.

Cardiovascular, respiratory and laboratory markers are included in Table 3.

TABLE 3
Cardiovascular, respiratory and laboratory markers in
Study 1 at baseline and after 5 days of treatment

| | I group | | II group | |
|---|---|---|---|---|
| | HIDEX | | SPIDEX | |
| | Baseline | After 5 d | Baseline | After 5 d |
| BP_{syst.}, mm | 139.6 ± 15.5 | 150.1 ± 20.1# | 137.5 ± 22.5 | 128.3 ± 12.0*# |
| Hg | | | | |
| BP_{diast.}, | 86 ± 8.6 | 89.0 ± 10.6 | 85.25 ± 12.40 | 81.1 ± 6.7*# |
| mm Hg | | | | |
| HR/min | 84.7 ± 10.8 | 89.1 ± 10.6 | 83.8 ± 11.8 | 73.0 ± 4.8*# |
| Breath | 24.8 ± 4.6 | 18.4 ± 3.1# | 24.2 ± 4.1 | 16.2 ± 2.4# |
| rate/min | | | | |
| Blood | 91.8 ± 3.6 | 94.1 ± 3.0 | 91.7 ± 3.3 | 95.5 ± 2.6 |
| oxygen, % | | | | |
| CRP, mg/l | 72.4 ± 61.7 | 28.1 ± 22.9# | 65.75 ± 50.9 | 17.1 ± 17.7*# |
| | (6.4-204) | (2.8-83.5) | (10.2-209) | (2.8-62.6) |
| D-dimer, | 736.7 ± 680.6 | 574.8 ± 306.4# | 755.5 ± 606.1 | 357.3 ± 271.1*# |
| ng/ml | (85-2500) | (38-2800) | (145-2500) | (105-1500) |
| Fasting | 5.9 ± 2.0 | 11.69 ± 6.21# | 6.89 ± 3.22 | 9.44 ± 2.94* |
| glucose, | | | | |
| mmol/l | | | | |

Figures expressed as Mean and Standard Deviation
#p < 0.05 in comparison with baseline;
*p < 0.05 in comparison with Group I

TABLE 4
Dynamics of radiological indicators on the background of treatment

| | I Group | II Group |
|---|---|---|
| Radiological data | HIDEX | SPIDEX |
| Signs of pneumonia at the beginning of treatment | 40 | 40 |
| Resorption of pneumonia on day 5 | 15 | 24 |
| Deterioration of the Chest X-ray picture assessed | 4 | 0 |
| on the 5th day | | |

Systolic blood pressure was significantly higher in the HIDEX group compared to baseline in comparison with the SPIDEX group where it was significantly lower. The respiratory rate was significantly reduced by both treatments. Blood oxygen saturation rose in both groups. C-reactive protein was significantly lowered by both treatment regimens but was significantly lower at 5 days in the SPIDEX than the HIDEX group. The same pattern was found for D-Dimer levels. The fasting blood glucose was significantly elevated in the HIDEX group but not in the SPIDEX group. The SPIDEX fasting blood glucose was significantly lower than that of the HIDEX.

### Radiological Findings

All 80 patients had radiological evidence of bilateral pneumonia. At 5 days there was radiological evidence of resorption of the pneumonia in 24 of the 40 patients in the SPIDEX group and 15 of the 40 in the HIDEX group. In 5 patients in the HIDEX group there was deterioration of the Chest X-ray on day⁵.

### Study 2

In this study of patients with a diagnosis of COVID-19 who were treated within 2-3 days of the onset of symptoms with low dose dexamethasone (4 mg/day) and spironolactone (100 mg/day) for 10 days (SPIDEX) only two patients required hospitalisation. The majority of patients showed major improvement after 5 days and over 90% were asymptomatic at 10 days. No patient became hyperkalaemic.

TABLE 5
Study 2. Clinical characteristics and plasma potassium levels before
and after 5 and 10 days on low dose dexamethasone and spironolactone
(SPIDEX) in 20 patients with positive COVID-19 PCR test where
treatment was started within 2-3 days of symptom onset.

| | | After 5 d | After 10 d |
|---|---|---|---|
| Complaints | Baseline | SPIDEX | SPIDEX |
| Loss of smell / taste | 18 (90%) | 8 (40%) | 1 (5%) |
| Sore throat | 11 (55%) | 3 (15%) | 0 |
| Breathlessness | 10 (50%) | 6 (30%) | 2 (10%) |
| Dry cough | 16 (80%) | 11 (55%) | 2 (10%) |
| Fever | 16 (80%) | 4 (20%) | 0 |
| Body temperature ° C. | 37.8 ± 0.7 | 36.8 ± 0.2* | 36.6 ± 0.2* |
| Blood oxygen < 92% | 0 | 2 | 0 |
| Oxygen saturation (%) | 96.4 ± 1.7 | 97.7 ± 2.9 | 98.6 ± 1.4 |
| Plasma potassium, mmol/l | 4.36 ± 0.68 | 5.04 ± 0.51 | 4.87 ± 0.45 |

### Interpretation

This work was designed to test the hypothesis that corona virus-induced exocytosis of ATP from epithelial cells and Weibel-Palade bodies from endothelial cells is important in the pathogenesis of SARS-CoV-2 infection. This process results from oxidative stress activation of the mineralocorticoid receptor. If, as suggested, exocytosis of ATP plays a key role in the genesis of loss of smell/taste, cough and the inflammatory response it might be predicted that this could be inhibited by mineralocorticoid receptor blockade. This is supported by the results showing that SPIDEX (spironolactone and 4 mg/day dexamethasone) produced major benefit with regard to recovery of taste/smell, cough and fever in comparison to HIDEX (dexamethasone 16 mg/day). This suggests that the major effect of dexamethasone may well be via suppression of cortisol secretion and hence its activation of the MR rather than acting as an anti-inflammatory agent via the glucocorticoid receptor.

Endothellitis plays a major role in the pathogenesis of the key complications of COVID-19, the Acute Respiratory Distress Syndrome and the genesis of microthrombi. The latter are 9 times more common in the lungs of patients dying from SARS-CoV-2 infection than with influenza related pneumonia. A key indicator of thrombosis is D-dimer. Early studies from Wuhan showed that D-dimer levels were the strongest independent predictor of mortality in COVID-19 patients. In Study 1, patients in both the HIDEX and the SPIDEX groups had a significant reduction in D-dimer levels. However, the levels in the SPIDEX treated patients were significantly lower than those on HIDEX. It is suggested that this is related to blockade of the exocytosis of Weibel-Palade bodies containing Von Willebrand Factor.

The high levels of co-morbidities in study 1 are typical of patients with severe COVID-19. These conditions all predispose to vascular disease which is key to the expression of the ACE2 receptors on vascular endothelium².

The more rapid resolution of pneumonia that was observed in study 1 SPIDEX patients might be expected if inhibition of endothelial exocytosis blocked the release of the capillary permeability factor angiopoietin². This appears to play an important role in the genesis of the Acute Respiratory Distress Syndrome (Kumpers, P. & Lukasz, A. The curse of angiopoietin-2 in ARDS: On stranger TI(E)des. Crit. Care 22, 4-7 (2018). Rising levels of angiopoietin are predictive of a poor outcome.

Study 2 was aimed attesting the effectiveness and safety of giving SPIRODEX to 20 outpatients within 2-3 days of the onset of symptoms. Only 2 of these patients required hospital admission within 10 days and that was brief. 30% had co-morbidities that might increase their potential for severe disease. There was marked improvement after 5 days and this was virtually complete after 10 days. At this time only 1 patient had loss of taste and smell compared to 18 pre-treatment. Two patients still had a dry cough and 2 were breathless. Of particular importance no patient had significant hyperkalaemia.

### EXAMPLE REFERENCES

2. Kaneko, N. et al. Flow-Mediated Susceptibility and Molecular Response of Cerebral
5. Jeong, Y. et al. Aldosterone activates endothelial exocytosis. Proc. Natl. Acad. Sci. U.S.A. 106, 3782-3787 (2009).

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise paragraphed. No language in the specification should be construed as indicating any non-paragraphed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the paragraphs appended hereto as permitted by applicable law.

1. A mineralocorticoid receptor antagonist, or a pharmaceutically acceptable salt thereof, for use in the prevention and/or treatment of a coronavirus infection, wherein the mineralocorticoid receptor antagonist, or a pharmaceutically acceptable salt thereof, reduces the viral load in the patient.

2. A mineralocorticoid receptor antagonist, or a pharmaceutically acceptable salt thereof, for use in the prevention and/or treatment of a coronavirus infection, wherein the mineralocorticoid receptor antagonist, or a pharmaceutically acceptable salt thereof, reduces the transmission of virus.

3. A mineralocorticoid receptor antagonist, or a pharmaceutically acceptable salt thereof, for use in the prevention of viral transmission in an individual infected with a virus (such as a coronavirus infection.

4. A mineralocorticoid receptor antagonist, or a pharmaceutically acceptable salt thereof, for use in the reduction of the viral load in an individual infected with a virus (such as a coronavirus infection.

5. A mineralocorticoid receptor antagonist, or a pharmaceutically acceptable salt thereof, for use in the prevention and/or treatment of pneumonia, anosmia, ageusia (loss of taste), and/or persistent cough, in a patient suffering with anosmia, ageusia (loss of taste), and/or persistent cough.

6. A mineralocorticoid receptor antagonist, or a pharmaceutically acceptable salt thereof, for the use claimed in claim 5, wherein the pneumonia, anosmia, ageusia (loss of taste), and/or persistent cough is caused by a coronavirus infection.

7. A mineralocorticoid receptor antagonist, or a pharmaceutically acceptable salt thereof, for the use claimed in any one of claims 1 to 6, wherein the coronavirus infections is COVID-19 (SARS-CoV-2).

8. A mineralocorticoid receptor antagonist, or a pharmaceutically acceptable salt thereof, for the use claimed in any one of claims 1 to 7, wherein the mineralocorticoid receptor antagonist, or a pharmaceutically acceptable salt thereof, is selected from spironolactone, eplerenone or canrenone.

9. A mineralocorticoid receptor antagonist, or a pharmaceutically acceptable salt thereof, for the use claimed in any one of claims 1 to 8, wherein the mineralocorticoid receptor antagonist, or a pharmaceutically acceptable salt thereof, is selected from spironolactone or eplerenone.

10. A mineralocorticoid receptor antagonist, or a pharmaceutically acceptable salt thereof, for the use claimed in any one of claims 1 to 9, wherein the mineralocorticoid receptor antagonist is spironolactone, or a pharmaceutically acceptable salt thereof.

11. A mineralocorticoid receptor antagonist, or a pharmaceutically acceptable salt thereof, for the use claimed in claim 10, wherein spironolactone is administered at a dosage within the range of 25 mg-400 mg per day.

12. A mineralocorticoid receptor antagonist, or a pharmaceutically acceptable salt thereof, for the use claimed in claim 10, wherein spironolactone is administered at a dosage within the range of 25 mg to 200 mg per day.

13. A mineralocorticoid receptor antagonist, or a pharmaceutically acceptable salt thereof, for the use claimed in claim 10, wherein at a dosage of 50 to 150 mg per day as a single dose or as a dosage of 25 mg to 75 mg twice daily.

14. A mineralocorticoid receptor antagonist, or a pharmaceutically acceptable salt thereof, for the use claimed in claim 10, wherein spironolactone is administered as a single daily dose of 100 mg or at a dosage of 50 mg twice daily.

15. A mineralocorticoid receptor antagonist, or a pharmaceutically acceptable salt thereof, for the use claimed in any one of claims 1 to 14, wherein the mineralocorticoid receptor antagonist is administered orally, nasally or to the buccal cavity.

16. A mineralocorticoid receptor antagonist, or a pharmaceutically acceptable salt thereof, for the use claimed in any one of claims 1 to 15, wherein the mineralocorticoid receptor antagonist is administered nasally or to the buccal cavity, optionally in the form of a spray.

17. A pharmaceutical composition comprising a mineralocorticoid receptor antagonist, or a pharmaceutically acceptable salt thereof, and one or more pharmaceutically acceptable excipients, and wherein the pharmaceutical composition is adapted for nasal and/or buccal delivery of the mineralocorticoid receptor antagonist.

18. A pharmaceutical composition according to claim 17, for a use as defined in any one of claims 1 to 16.

19. A pharmaceutical composition according to claim 17 or claim 18, wherein the pharmaceutical composition is in the form of a nasal spray, oral spray, nose drops or buccal tablet.

20. A pharmaceutical composition according to any one of claims 17 to 19, wherein the pharmaceutical composition is in the form of a nasal spray, oral spray, nose drops or buccal tablet.

21. A pharmaceutical composition according to any one of claims 17 to 19, wherein the mineralocorticoid receptor antagonist is spironolactone.

22. A method of:
(i) preventing and/or treating a coronavirus infection (e.g. COVID-19);
(ii) preventing viral transmission in an individual infected with a virus (such as a coronavirus infection (e.g. COVID-19),
(iii) reducing viral load in an individual infected with a virus (such as a coronavirus infection (e.g. COVID-19)
(iv) preventing and/or treating anosmia, ageusia (loss of taste), and/or persistent cough, in a patient suffering with anosmia, ageusia (loss of taste), and/or persistent cough (e.g. as a consequence of a coronavirus/COVID-19 infection)
the method comprising administering a therapeutically effective amount of a mineralocorticoid receptor antagonist, or a pharmaceutically acceptable salt thereof, to a patient in need of such treatment.
